(19) United States
(12) Patent Application Publication
(10) Pub. No.: US 2011/0009857 A1
(43) Pub. Date: Jan. 13, 2011

(54) OPEN-IRRIGATED ABLATION CATHETER WITH TURBULENT FLOW

(76) Inventors: Raj Subramaniam, Fremont, CA (US); Josef Koblish, Sunnyvale, CA (US); Mark Mirigian, Hayward, CA (US)

Correspondence Address: Schwegman Lundberg & Woessner/ EPT-CRM;EPT-CRM, P.O. Box 2938, Minneapolis, MN, 55402, US

(21) Appl. No.: 12/834,265

(22) Filed: Jul. 12, 2010

Related U.S. Application Data
(60) Provisional application No. 61/225,118, filed on Jul. 13, 2009.

(51) Int. Cl. A61B 18/12; A61B 18/18; H01R 43/00

(52) U.S. Cl. 606/33; 606/41; 29/825

(57) ABSTRACT

According to an embodiment of a method for cooling an open-irrigated ablation electrode, pressurized fluid is delivered from a fluid lumen of a catheter body into an ablation electrode. Fluid flow in the fluid lumen is generally laminar. The generally laminar fluid flow is transformed from the fluid lumen into a turbulent fluid flow within the ablation electrode. The pressurized fluid with turbulent fluid flow is delivered through irrigation ports of the ablation electrode.

### TECHNICAL FIELD

This application claims the benefit of U.S. Provisional Application No. 61/225,118, filed on Jul. 13, 2009, under 35 U.S.C. §119(e), which is hereby incorporated by reference in its entirety.

### TECHNICAL FIELD

This application relates generally to medical devices and, more particularly, to systems and methods related to open-irrigated ablation catheters.

### BACKGROUND

Aberrant conductive pathways disrupt the normal path of the heart's electrical impulses. For example, conduction blocks can cause the electrical impulse to degenerate into several circular wavelets that disrupt the normal activation of the atria or ventricles. The aberrant conductive pathways create abnormal, irregular, and sometimes life-threatening heart rhythms called arrhythmias. Ablation is one way of treating arrhythmias and restoring normal contraction. The sources of the aberrant pathways (called focal arrhythmia substrates) are located or mapped using mapping electrodes. After mapping, the physician may ablate the aberrant tissue. In radio frequency (RF) ablation, RF energy is directed from the ablation electrode through tissue to ablate the tissue and form a lesion.

Heat is generated during the RF ablation process, and this heat may cause a thrombus (blood clot). Some ablation catheter systems have been designed to cool the electrode and surrounding tissue. For example, open-irrigated catheter systems pump a cooling fluid, such as a saline solution, through a lumen in the body of the catheter, out through the ablation electrode, and into surrounding tissue. The cooling fluid cools the ablation electrode and surrounding tissue, thus reducing the likelihood of a thrombus, preventing or reducing impedance rise of tissue in contact with the electrode tip, and increasing energy transfer to the tissue because of the lower tissue impedance.

### SUMMARY

An embodiment of an open-irrigated ablation catheter system comprises a catheter body, a generally hollow electrode tip body, and a distal insert. The catheter body has a fluid lumen. The electrode tip body has a closed distal end and an open proximal end for connection to the catheter body. The electrode tip body has a plurality of irrigation ports to enable fluid to exit from the electrode tip body. The distal insert is positioned in the electrode tip body to define a proximal fluid chamber and a distal fluid chamber in the electrode tip body. The distal insert has a fluid conduit between the proximal fluid chamber and the distal fluid chamber. The plurality of irrigation ports enable fluid to exit from the distal fluid chamber. The electrode tip body and the distal insert are configured to enable pressurized fluid to flow from the fluid lumen in the catheter body into the proximal fluid chamber, from the proximal fluid chamber into the fluid conduit, from the fluid conduit into the distal fluid chamber; and from the distal fluid chamber through the plurality of irrigation ports.

According to an embodiment of a method for forming an open-irrigated ablation electrode tip, a generally cylindrical electrode tip body is formed. A distal end of the electrode tip body is a closed end and a proximal end of the electrode tip body is an open end. Irrigation ports are formed around a circumference of the electrode tip body proximate to the distal end of the electrode tip body. The irrigation ports allow fluid to flow out from within the electrode tip body. A distal insert is placed in the generally cylindrical tip body. A distal fluid chamber reservoir is defined by the distal insert and the electrode tip body. The distal fluid chamber is between the distal end of the electrode tip body and the distal insert. The electrode tip body is connected to a catheter body. A proximal fluid chamber is defined by the distal insert and the electrode tip body. The distal insert includes a fluid conduit extending between the proximal fluid chamber to the distal fluid chamber.

According to an embodiment of a method for cooling an open-irrigated ablation electrode, pressurized fluid is delivered from a fluid lumen of a catheter body into an ablation electrode. Fluid flow in the fluid lumen is generally laminar. The generally laminar fluid flow is transformed from the fluid lumen into a turbulent fluid flow within the ablation electrode. The pressurized fluid with turbulent fluid flow is delivered through irrigation ports of the ablation electrode.

This Summary is an overview of some of the teachings of the present application and not intended to be an exclusive or exhaustive treatment of the present subject matter. Further details about the present subject matter are found in the detailed description and appended claims. The scope of the present invention is defined by the appended claims and their equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1A illustrates an open-irrigated catheter electrode tip with near laminar flow of cooling fluid at the exit ports; and FIG. 1B illustrates an open-irrigated catheter electrode tip designed to cause the cooling fluid to exit the electrode with a turbulent flow.

FIG. 2 illustrates an ablation electrode designed to promote turbulent flow, according to an embodiment of the present subject matter.

FIGS. 3A-D illustrate various views of an embodiment of an ablation electrode tip.

FIG. 4 illustrates an electrode tip embodiment with a distal insert illustrated therein.

FIG. 5A-5B illustrate planar and cross-sectional views of an embodiment of a distal insert.

FIGS. 6A-6D illustrate a process for manufacturing the electrode, according to various embodiments, and further illustrate the turbulent flow generated by the electrode design.

FIG. 7 illustrates an embodiment of a mapping and ablation system, wherein the system includes an open-irrigated catheter that promotes turbulent flow, according to various embodiments of the present subject matter.

### DETAILED DESCRIPTION

The following detailed description of the present invention refers to subject matter in the accompanying drawings which show, by way of illustration, specific aspects and embodiments in which the present subject matter may be practiced. These embodiments are described in sufficient detail to enable those skilled in the art to practice the present subject matter. References to “an,” “one,” or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

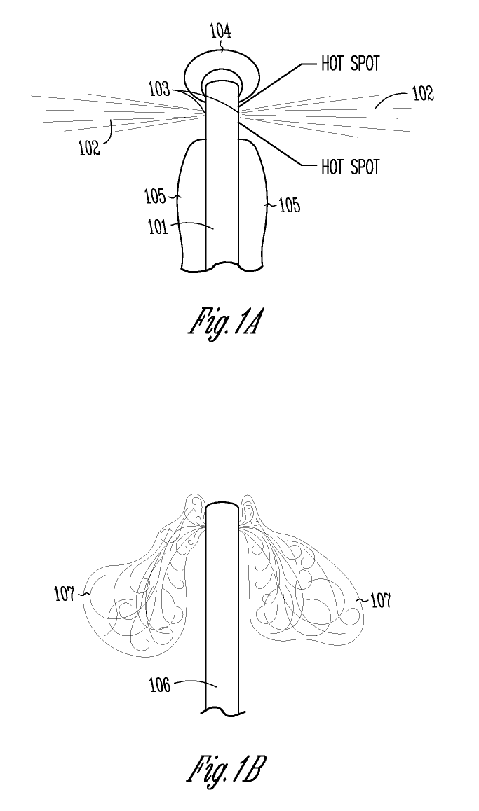

If near laminar flow conditions are at the exit ports of an open-irrigated catheter, stable eddy currents may be formed around the electrode. Under these conditions, there could be hot spots by the ablation electrode, particularly around the proximal part of the electrode. If these stable eddy currents trap blood platelets near the electrode, and if these trapped platelets are activated due to heat and shear force, a thrombus could potentially form. FIG. 1A illustrates an open-irrigated catheter electrode tip 101 with near laminar flow of cooling fluid 102 at the exit ports 103, also referred to as irrigation ports. The figure illustrates a cross sectional view around the electrode tip, and does not illustrate cooling jets outside of the plane of the figure. The cooling fluid within this electrode tends to cool the tissue at the distal end as represented by the cooled area 104, and toward the proximal end, as represented by the cooled area 105. The near laminar flow of cooling fluid 102 from the irrigation ports 103 tends to cause the cooling fluid to flow away from the ablation electrode and the tissue near the ablation site, potentially causing uneven cooling and localized hot spots along the ablation electrode.

The present subject matter provides systems and methods for cooling the ablation electrode and the surrounding tissue in a more uniform manner. An open-irrigated RF ablation catheter is designed to promote turbulent flow of cooling fluid both within and outside of the electrode to improve the uniformity of cooling. FIG. 1B illustrates an open-irrigated catheter electrode tip 106 designed to cause the cooling fluid to exit the electrode with a turbulent flow 107. The figure illustrates a cross sectional view around the electrode tip, and does not illustrate cooling jets outside of the plane of the figure. The turbulence around the body of the electrode tip 106 encourages a more uniform cooling of the electrode body, and also encourages the dilution of the blood in the vicinity of the ablation electrode. The risk of thrombus formation significantly decreases using turbulent flow of cooling fluid to both dilute the blood near the electrode and uniformly cool the electrode.

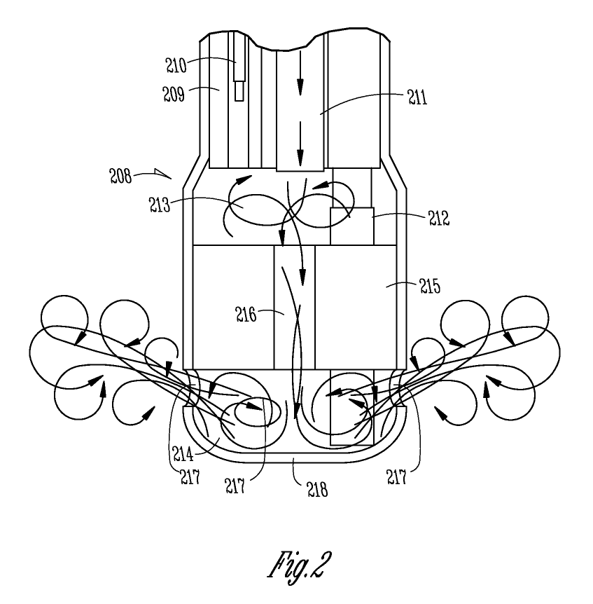

FIG. 2 illustrates an ablation electrode 208, according to an embodiment of the present subject matter, designed to promote turbulent flow. The electrode 208 is designed to thoroughly mix the cooling fluid in the surrounding tissue close to and around the ablation electrode. The ablation electrode 208 is connected to a catheter body 209, which has a lumen therein to receive an RF wire 210 used to deliver the RF energy from an RF generator to the RF electrode. The catheter body 209 also has a cooling lumen 211 used to deliver pressurized cooling fluid from a coolant reservoir to the ablation electrode 208. A thermocouple 212 is also delivered through a lumen in the catheter body. The ablation electrode 208 includes a proximal chamber 213 and a distal chamber 214 separated by a distal insert 215. The distal insert 215 includes a fluid conduit 216 connecting the proximal and distal chambers, and further includes an opening to receive the thermocouple 212, allowing a distal end of the thermocouple to be positioned in the distal chamber 214. Exit or irrigation ports 217 provide openings through which the cooling fluid flows from the distal chamber 214 to the outside of the ablation electrode 208.

The proximal and distal chambers 213 and 214, the cooling lumen 211, the fluid conduit 216, and the irrigation ports 217 are designed with appropriate dimensions and geometry with respect to each other to encourage turbulent fluid flow when pressurized cooling fluid flows out of the cooling lumen 211 in the catheter, through the proximal chamber 213, through the fluid conduit 216 in the distal tip insert, through the distal chamber 214 and out the irrigation ports 217. Coolant is pumped at high pressure in the catheter. When it enters the proximal chamber of the electrode tip, the fluid circulates within the chamber to cool the proximal electrode and mitigate overheating (edge effect). Laminar flow is further disturbed as the coolant is forced in to the distal chamber. The turbulence increases as the coolant exits through the irrigation ports in the tip electrode. The edges of the irrigation ports are purposely left rough and ragged. The distal end 218 of the distal chamber is a relatively flat wall to further encourage the laminar flow of the pressurized fluid as it flows through the fluid conduit in the distal tip insert. The combination of these factors causes the fluid exiting the coolant port to create turbulence around the entire electrode body, encouraging a more uniform cooling of the electrode body and the dilution of the blood in the vicinity of the ablation electrode. Additionally, in the illustrated embodiment, the arrangement of the irrigation ports with respect to the distal chamber encourages the fluid to flow out at an angle toward the proximal end of the ablation electrode to cause the cooling fluid to flow, in a turbulent manner, at the proximal end of the electrode as well as at the distal end of the electrode.

FIGS. 3A-D illustrate various views of an ablation electrode tip, according to various embodiments. The illustrated electrode tip 308 has six irrigation ports 317 equally spaced around the circumference of the electrode tip, such that each port is approximately 60 degrees away from the adjacent ports. The present subject matter is not limited to equally-spaced irrigation ports or to a particular number of irrigation ports. The system can be designed with other numbers and arrangements of irrigation ports. FIG. 3A illustrates the electrode tip 308 at a first rotational position with respect to an axis of rotation 319, and FIG. 3B illustrates the electrode tip 308 at a second rotational position with respect to the axis 319, wherein the second rotational position is approximately 30 degrees from the first rotational position. FIG. 3C illustrates a view from a proximal end of the electrode tip, and FIG. 3D illustrates a cross-sectional view of the electrode tip taken along line A-A in FIG. 3C.

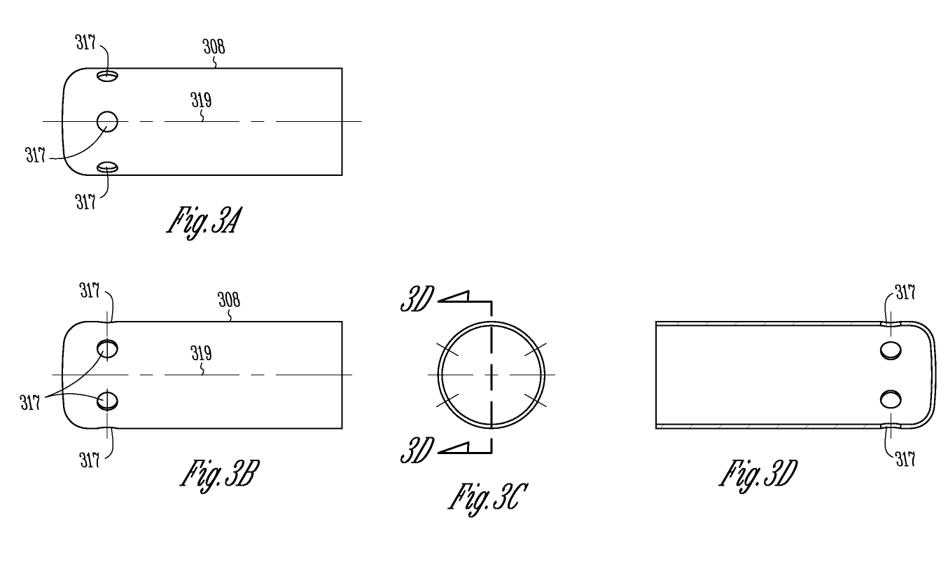

FIG. 4 illustrates an electrode tip embodiment 408 with a distal insert 415 illustrated therein. The illustrated electrode 408 includes a generally flat distal end 418. The illustrated electrode 408 also includes a proximal portion 421 and a distal portion 422. In the illustrated embodiment, the distal portion 422 has a generally cylindrical configuration with a first diameter, and the proximal portion 421 has a generally cylindrical configuration with a second diameter reduced from the first diameter. The distal insert, the distal fluid chamber, and the proximal fluid chamber are within the distal portion of the electrode. The irrigation ports 417 are on the distal portion 422. The illustrated distal insert includes a fluid conduit 416 extending between the proximal and distal chambers, and further includes a thermocouple opening 420 configured to receive a thermocouple to allow a distal end of the thermocouple to be placed in the distal chamber of the electrode 408.

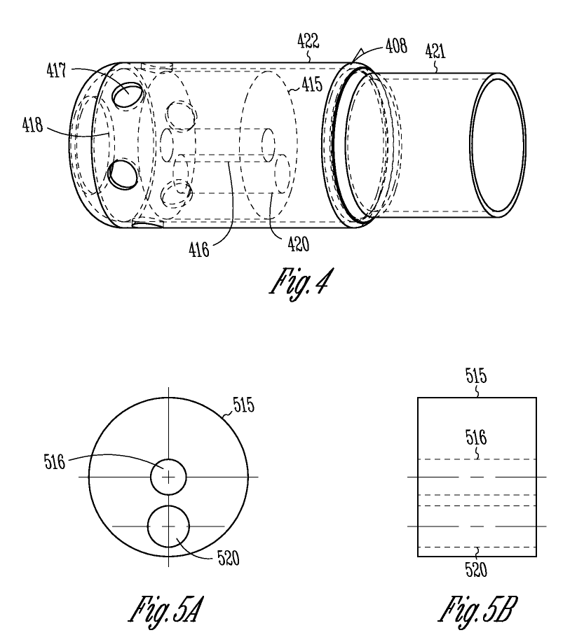

FIG. 5A illustrates a planar view of the distal insert 515, and FIG. 5B illustrates a cross-sectional view of the distal insert. These views further illustrate the fluid conduit 516 and the thermocouple opening 520 formed in the distal insert.

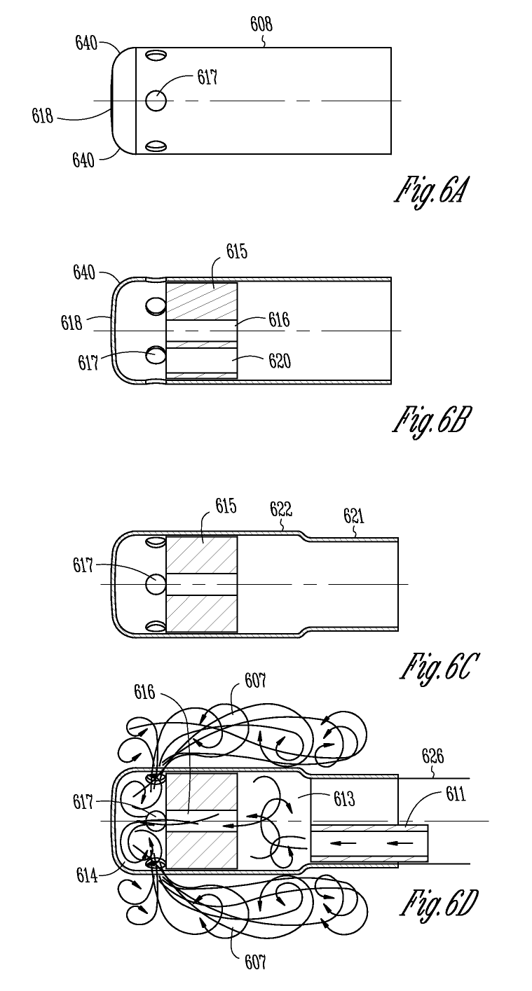

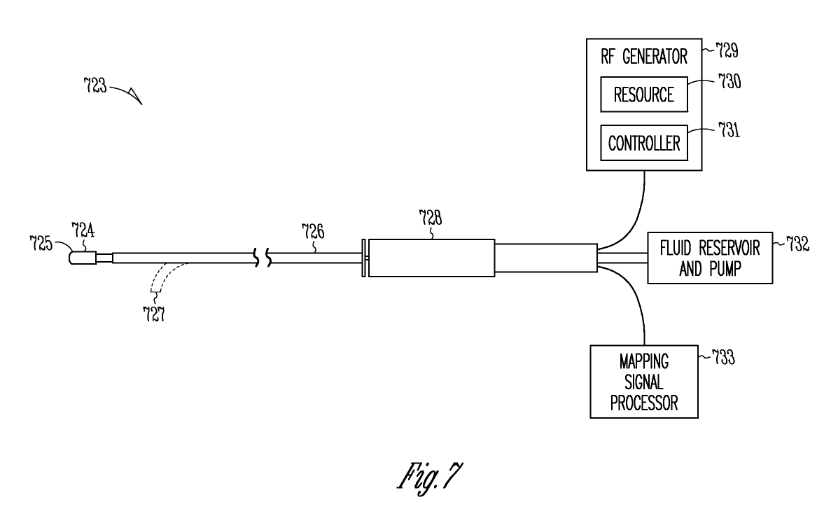

FIGS. 6A-6D illustrate a process for manufacturing the electrode, according to various embodiments, and further illustrate the turbulent flow generated by the electrode design. FIG. 6A illustrates the electrode 608 after the tip is drawn and the irrigation ports 617 are drilled or otherwise formed. For example, some embodiments form the irrigation ports with a spark EDM (electric discharge machining) process. The edges of the irrigation ports are intended to be rough. By way of example, the edges are not machined smooth after the ports are formed. The hollow tip body has an open interior region defined by an exterior wall of the tip section. In the illustrated embodiment, the hollow tip body has a generally cylindrical shape. The distal end 618 is generally flat (e.g. a slight curvature) with rounded edges 640.

FIG. 6B illustrates a cross-sectional view of the electrode tip after the distal insert 615 is placed inside the electrode tip. As illustrated in FIG. 6B, the distal insert 615 is positioned against the rough edges of the irrigation ports 617. By way of an example and not limitation, an embodiment of the electrode tip body has a diameter on the order of about 0.08-0.1 inches, has a length on the order of about 0.2-0.3 inches, and has an exterior wall with a thickness on the order of 0.003-0.004 inches. The distal insert 615 has a diameter corresponding to the interior diameter of the electrode tip. For example, in an embodiment, the distal insert has a diameter of approximately 0.08 inches and a width of approximately 0.06 inches. The fluid conduit 616 has a diameter between approximately 0.015 to 0.020 inches. The thermocouple opening 620 is sized to receive a thermocouple. In the illustrated embodiment, the thermocouple opening is approximately 0.02 inches. The tip section 102 is formed from a conductive material. For example, some embodiments use a platinum-iridium alloy. Some embodiments use an alloy with approximately 90% platinum and 10% iridium. This conductive material is used to conduct RF energy used to form legions during the ablation procedure. A plurality of irrigation ports 617 or exit ports are shown near the distal end of the tip section. By way of example and not limitation, an embodiment has irrigation ports with a diameter approximately within a range of 0.01 to 0.02 inches. Thus, according to various embodiments, the irrigation portions have a width (represented by the diameter of the ports of approximately 0.01 to approximately 0.02) to height (represented by the wall thickness of the electrode tip body of approximately 0.003 to approximately 0.004) ratio between about 2 to 7. This ratio may be referred to as an aspect ratio of the irrigation ports. Larger aspect ratios in comparison to small aspect ratios promote more turbulent flow. The irrigation ports 617 are formed approximately 0.30 to 0.45 inches from the distal end of the electrode tip. The distal end 618 is generally flat. For example, in an embodiment in which the outer diameter of the electrode is approximately 0.09 inches, the curvature of the relatively flat distal portion 618 has a radius of approximately 0.29 inches. In the illustrated embodiment, the radius of the edges 640 at the distal end is approximately 0.02 inches. Fluid, such as a saline solution, flows through these ports 617 to the exterior of the electrode. This fluid is used to cool the ablation electrode tip and the tissue near the electrode. This temperature control reduces coagulum formation on the tip of the catheter, prevents impedance rise of tissue in contact with the catheter tip, and increases energy transfer to the tissue because of the lower tissue impedance.

FIG. 6C illustrates the electrode after the proximal portion 621 of the electrode tip is swaged. According to various embodiments, the length of the distal portion is approximately 0.15 to 0.17 inches. In some embodiments, the length of the distal portion is between 0.155 to 0.160 inches. The proximal portion has a length, in some embodiments, of approximately 0.06 to 0.08 inches. In some embodiments, the length of the proximal portion is between 0.070 to 0.075 inches. In the illustrated embodiment, the diameter of the distal portion is approximately 0.09 inches and the diameter of the proximal portion is approximately 0.07 to 0.08 inches.

FIG. 6D illustrates a cooling lumen 611 of the catheter, after the electrode is connected to the catheter body 626. The cooling lumen 611 of the catheter may have a diameter of, by way of example, 0.02 to 0.04 inches, which corresponds to a cross-sectional area of about 0.0006 in² to about 0.003 in². Various catheter embodiments include more than one lumen. For example, some catheter embodiments have a dual lumen structure, where the structure has two side-by-side channels. By way of example and not limitation, the diameter of each lumen in one dual lumen structure embodiment is approximately 0.019 inches, where the combination of lumens provide a total cross-sectional area of about 0.0011 in² (about 0.00057 in² for each lumen).

FIG. 6D further illustrates fluid flowing through the cooling lumen into the proximal chamber 613. The diameter of the fluid passage expands significantly from the cooling lumen 611 (e.g. 0.03 inches) to the inner diameter of the proximal chamber (e.g. 0.08 inches). The diameter of the fluid passage retracts significantly from the proximal chamber (e.g. 0.09 inches) to the fluid conduit 616 (0.018 inches) of the distal insert. These changes in the fluid passage cause the pressurized fluid to circulate or mix in the proximal chamber before proceeding through the fluid conduit 616 of the distal insert to the distal chamber 614. In the illustrated embodiment, the length of the proximal fluid chamber 613 is approximately 0.06 inches, corresponding to the width of the digital insert. The diameter of the fluid passage expands again from the fluid conduit 616 (e.g. 0.018 inches) to the inner diameter of the distal chamber (e.g. 0.08 inches), which further encourages the pressurized fluid to circulate or mix. In the illustrated embodiment, the length of the distal fluid chamber 614 is approximately 0.04 inches. Additionally, the pressurized fluid deflects off of the distal wall of the electrode to further mix the fluid, and to cause the fluid to exit out of the irrigation ports 617 toward the proximal end of the electrode. The mixing of the pressurized fluid within the electrode changes the laminar flow of the fluid within the cooling lumen 611 of the catheter into a turbulent flow, represented at 607, as the fluid exits the irrigation ports. The irrigation portions are relatively large (diameter of approximately 0.017 inches), and the electrode walls of the irrigation ports are relatively thin (e.g. 0.003 inches). This geometry, in addition to the rough edges of the irrigation portions, further encourages the turbulent nature of the fluid flow as it exits the distal chamber. The geometry of the proximal chamber 613, the distal chamber 614, the fluid conduit 616, and the irrigation ports 617 can be adjusted to change the fluid flow characteristics of the pressurized fluid as it exits the irrigation ports.

FIG. 7 illustrates an embodiment of a mapping and ablation system 723, wherein the system includes an open-irrigated catheter that promotes turbulent flow, according to various embodiments of the present subject matter. The illustrated catheter includes an ablation tip 724 with an RF ablation electrode 725 and irrigation ports therein. The catheter can be functionally divided into four regions: the operative distal ablation electrode 725, a main catheter region 726, a deflectable catheter region 727, and a proximal catheter handle region where a handle assembly 728 including a handle is attached. A body of the catheter includes a cooling fluid lumen and may include other tubular element(s) to provide the desired functionality to the catheter. The addition of metal in the form of a braided mesh layer sandwiched in between layers of plastic tubing may be used to increase the rotational stiffness of the catheter.

The deflectable catheter region 727 allows the catheter to be steered through the vasculature of the patient and allows the probe assembly to be accurately placed adjacent the targeted tissue region. A steering wire (not shown) may be slidably disposed within the catheter body. The handle assembly may include a steering member to push and pull the steering wire. Pulling the steering wire causes the wire to move proximally relative to the catheter body which, in turn, tensions the steering wire, thus pulling and bending the catheter deflectable region into an arc. Pushing the steering wire causes the steering wire to move distally relative to the catheter body which, in turn, relaxes the steering wire, thus allowing the catheter to return toward its form. To assist in the deflection of the catheter, the deflectable catheter region may be made of a lower durometer plastic than the main catheter region.

The illustrated system 723 includes an RF generator 729 used to generate the energy for the ablation procedure. The RF generator 729 includes a source 730 for the RF energy and a controller 731 for controlling the timing and the level of the RF energy delivered through the ablation tip 724. The illustrated system 723 also includes a fluid reservoir and pump 732 for pumping cooling fluid, such as a saline, through the catheter and out through the irrigation ports. Some system embodiments incorporate a mapping function. Mapping electrodes may be incorporated into the catheter system. In such systems, a mapping signal processor 733 is connected to the mapping electrodes to detect electrical activity of the heart. This electrical activity is evaluated to analyze an arrhythmia and to determine where to deliver the ablation energy as a therapy for the arrhythmia. One of ordinary skill in the art will understand that the modules and other circuitry shown and described herein can be implemented using software, hardware, and/or firmware. Various disclosed methods may be implemented as a set of instructions contained on a computer-accessible medium capable of directing a processor to perform the respective method.

This application is intended to cover adaptations or variations of the present subject matter. It is to be understood that the above description is intended to be illustrative, and not restrictive. The scope of the present subject matter should be determined with reference to the appended claims, along with the full scope of legal equivalents to which such claims are entitled.

What is claimed is:

1. An open-irrigated ablation catheter system, comprising:
a catheter body with a fluid lumen therein;
a generally hollow electrode tip body with a closed distal end and an open proximal end for connection to the catheter body, wherein the electrode tip body has a plurality of irrigation ports to enable fluid to exit from the electrode tip body; and
a distal insert positioned in the electrode tip body to define a proximal fluid chamber and a distal fluid chamber in the electrode tip body, the distal insert having a fluid conduit between the proximal fluid chamber and the distal fluid chamber, wherein the plurality of irrigation ports enable fluid to exit from the distal fluid chamber;
wherein the electrode tip body and the distal insert are configured to enable pressurized fluid to flow from the fluid lumen in the catheter body into the proximal fluid chamber, from the proximal fluid chamber into the fluid conduit, from the fluid conduit into the distal fluid chamber, and from the distal fluid chamber through the plurality of irrigation ports.

2. The system of claim 1, wherein the irrigation ports have rough edges.

3. The system of claim 1, wherein the electrode tip body has a circumference, and the irrigation ports are approximately equally spaced about the circumference of the electrode tip body.

4. The system of claim 1, wherein the irrigation ports are proximate to the distal insert to enable fluid to exit the distal fluid chamber near the distal insert toward a proximal end of the distal fluid chamber.

5. The system of claim 4, wherein:
the electrode tip body has a proximal portion and a distal portion;
the distal portion includes the distal fluid chamber, the proximal fluid chamber, and the distal insert; and
the proximal portion is swaged to a reduced diameter with respect to the distal portion.

6. The system of claim 1, wherein:
each of the fluid lumen, the proximal fluid chamber, the fluid conduit, and the distal fluid chamber have a diameter;
the diameter of the proximal fluid chamber is larger than the diameter of the fluid lumen;
the diameter of the fluid conduit is smaller than the diameter of the proximal fluid chamber; and
the diameter of the distal fluid chamber is larger than the diameter of the fluid conduit.

7. The system of claim 1, wherein:
the proximal fluid chamber has a diameter of approximately 0.08 inches and a length of approximately 0.06;
the fluid conduit has a diameter of approximately 0.018 inches and a length of approximately 0.06 inches; and
the distal fluid chamber has a diameter of approximately 0.08 inches and a length of approximately 0.04 inches.

8. The system of claim 1, wherein the electrode tip body has an exterior wall with a thickness of approximately 0.003-0.004 inches, each irrigation port is formed in the exterior wall, and each irrigation port has a diameter of approximately 0.01 to 0.02 inches.

9. The system of claim 8, wherein six irrigation ports are approximately equally spaced about a circumference of the electrode tip body.

10. The system of claim 1, further comprising a fluid reservoir configured to deliver pressurized cooling fluid through the fluid lumen in the catheter body to the electrode tip body.

11. The system of claim 1, further comprising a radio frequency (RF) generator electrically connected to the electrode tip body to deliver RF ablation energy from the electrode tip body.

12. A method for forming an open-irrigated ablation electrode tip, comprising:
forming a generally cylindrical electrode tip body, wherein a distal end of the electrode tip body is a closed end and a proximal end of the electrode tip body is an open end;
forming irrigation ports around a circumference of the electrode tip body proximate to the distal end of the electrode tip body, wherein the irrigation ports allow fluid to flow out from within the electrode tip body;
placing a distal insert in the generally cylindrical tip body, wherein a distal fluid chamber reservoir is defined by the distal insert and the electrode tip body, and the distal fluid chamber is between the distal end of the electrode tip body and the distal insert; and
connecting the electrode tip body to a catheter body, wherein a proximal fluid chamber is defined by the distal insert and the electrode tip body, wherein the distal insert includes a fluid conduit extending between the proximal fluid chamber to the distal fluid chamber.

13. The method of claim 12, wherein forming the generally cylindrical electrode tip body includes drawing the electrode tip body.

14. The method of claim 12, wherein forming irrigation portions includes drilling irrigation ports, and leaving the irrigation ports rough.

15. The method of claim 12, wherein forming irrigation ports includes performing a spark EDM (electric discharge machining) process to form the irrigation ports, and leaving the irrigation ports rough.

16. The method of claim 12, wherein forming irrigation ports includes spacing the irrigation ports approximately equally around a circumference of the electrode tip body.

17. The method of claim 12, wherein connecting the electrode tip body to the catheter body includes swaging a proximal portion of the electrode tip body.

18. A method for cooling an open-irrigated ablation electrode, comprising:
delivering pressurized fluid from a fluid lumen of a catheter body into an ablation electrode, wherein fluid flow in the fluid lumen is generally laminar;
transforming the generally laminar fluid flow from the fluid lumen into a turbulent fluid flow within the ablation electrode; and
delivering the pressurized fluid with turbulent fluid flow through irrigation ports of the ablation electrode.

19. The method of claim 18, wherein transforming the generally laminar fluid flow into the turbulent fluid flow includes:
receiving the pressurized fluid from the fluid lumen of the catheter body into a proximal fluid chamber, wherein a diameter of the proximal fluid chamber is larger than a diameter of the fluid lumen;
receiving the pressurized fluid from the proximal fluid chamber into a fluid conduit, wherein a diameter of the fluid conduit is smaller than a diameter of the proximal fluid chamber; and
receiving the pressurized fluid from the fluid conduit into a distal fluid chamber, wherein a diameter of the distal fluid chamber is larger than the diameter of the fluid conduit.

20. The method of claim 18, wherein delivering generally turbulent fluid through irrigation ports includes delivering fluid through irrigation ports that are not machined smooth after the irrigation ports are formed.

21. The method of claim 18, wherein delivering generally turbulent fluid through irrigation ports includes directing fluid flow out from the electrode and toward a proximal end of the electrode.
